(19) United States
(12) Patent Application Publication
(10) Pub. No.: US 2026/0366966 A1
(43) Pub. Date: Nov. 19, 2026

(54) Self-Actuating, Fail-Safe Inline Check Valve Assembly for Air Sampling Pipe Network Purging and Monitoring

(71) Applicant: Steven William Joseph, Portland, OR (US)

(72) Inventor: Steven William Joseph, Portland, OR (US)

(21) Appl. No.: 19/649,521

(22) Filed: Apr. 16, 2026

Related U.S. Application Data
(60) Provisional application No. 63/807,592, filed on May 17, 2025.

(51) Int. Cl. G01N 1/22; G01N 1/26; G01N 33/00; G08B 17/10

(52) U.S. Cl. CPC G01N 1/2247; G01N 1/26; G01N 33/007; G08B 17/10; G01N 2001/227

(57) ABSTRACT

A fluid-actuated, fail-safe inline check valve assembly for air sampling pipe networks is disclosed. The assembly includes a valve body configured for inline installation between an air sampling pipe and an air sampling smoke detection (ASD) unit, and a piston disposed within the valve body and biased by a spring element toward a default position permitting airflow to the ASD unit. A purge inlet is configured to receive pressurized fluid, which, upon exceeding a threshold pressure, displaces the piston to a purge position that closes an airflow pathway to the ASD unit and opens a purge pathway through the pipe network, thereby isolating the ASD unit while directing purge flow through the pipe network. Upon removal of the pressurized fluid, the piston automatically returns to the default position, restoring airflow to the ASD unit.

### TECHNICAL FIELD OF THE INVENTION

The present invention relates generally to air sampling smoke detection systems and associated piping networks, and more particularly to a self-actuating inline check valve assembly configured to facilitate automatic purging of air sampling pipe networks and sample holes while maintaining fail-safe operation, system integrity, and monitoring capability.

### BACKGROUND OF THE INVENTION

Air sampling smoke detectors (ASDs) are widely used in critical environments, including data centers, cold storage facilities, warehouses, industrial installations, and commercial buildings. These systems operate by drawing air through a network of pipes having distributed sample holes, with the collected air delivered to a central sensing unit for detection of smoke or other particulates. Over time, contaminants such as dust, lint, and airborne debris can accumulate within the pipe network and at the sample holes, thereby restricting airflow and degrading the performance and sensitivity of the detection system. In certain environments, such as cold storage applications, moisture accumulation may further lead to the formation of ice plugs within the piping, creating additional obstructions.

Conventional approaches for cleaning such pipe networks and sample holes typically involve manual procedures or the use of electrically actuated valve systems. In one common configuration, a manually operated ball valve is installed upstream of the ASD unit to isolate the detector from the pipe network during cleaning operations. Compressed air or cleaning fluid is then introduced through a dedicated port to backflush the pipe network and remove accumulated contaminants. However, such approaches present several drawbacks. Manual valves may be inadvertently left partially or fully open following maintenance, thereby compromising system functionality. Electrically actuated valves, such as solenoid-operated valves, increase system complexity, cost, and installation requirements. In systems lacking automated valves, routine maintenance requires direct human intervention, limiting the ability to automate cleaning operations.

Additionally, existing systems may present safety concerns. Following completion of a purge cycle, if the system is not fully depressurized, opening a valve while residual pressure remains in the piping can expose the ASD unit to unsafe air pressure conditions. These limitations highlight the need for an improved solution that enables effective purging of air sampling pipe networks while maintaining system integrity, reducing reliance on manual or electrically controlled components, and improving overall safety and reliability.

### SUMMARY OF THE INVENTION

The present invention provides a self-actuating, fail-safe inline check valve assembly configured for use in air sampling pipe networks associated with air sampling smoke detection (ASD) systems. The assembly is configured to facilitate automatic purging of pipe networks and sample holes while maintaining system integrity and protecting the ASD unit from exposure to purge pressure. As used herein, ‘pressurized fluid’ includes gases and liquids, including compressed air, inert gases, water, and other fluids capable of actuating the piston.

In one aspect, the invention includes a valve body configured for inline installation between an air sampling pipe and an ASD unit. A traveling piston is disposed within the valve body and is biased by a spring, which may comprise a compression spring or other biasing element, toward a default position in which an airflow pathway between the air sampling pipe and the ASD unit is open. A fluid inlet is provided to receive pressurized fluid at or above a threshold pressure sufficient to actuate the traveling piston.

Upon introduction of pressurized fluid through the fluid inlet, the traveling piston is displaced against the bias of the compression spring to a purge position in which movement of the traveling piston simultaneously closes the airflow pathway and opens the purge pathway. In this configuration, the ASD unit is isolated from the pipe network while pressurized fluid is directed through the pipe network to perform purging. Upon removal of the applied pressure, the compression spring returns the traveling piston to the default position, thereby restoring airflow to the ASD unit and terminating the purge pathway.

In certain embodiments, the valve assembly further includes one or more O-ring seals to maintain system integrity, alignment tabs to guide movement of the traveling piston, inlet and outlet inserts for interfacing with the pipe network, and union fittings to facilitate installation and servicing. The assembly may further include an RFID chip and antenna configured to store and track system data. An optional inline filter may be provided within the inlet or outlet.

The disclosed assembly provides a fail-safe configuration that automatically returns to normal operation following a purge cycle without requiring manual or electrically actuated components, thereby reducing system complexity, minimizing maintenance requirements, and preventing improper system configuration. The design is adaptable to various materials, pipe sizes, and operating environments, and may be applied in a range of facilities including data centers, warehouses, cold storage, industrial, and commercial environments. Although described in connection with air sampling smoke detection systems, the valve assembly may be used in any fluid conduit system in which selective isolation of a downstream component and redirection of fluid flow is desired, including industrial piping systems, fluid transport systems, HVAC systems, and process control systems.

### REFERENCE CHARACTERS AND PARTS LIST

101: Valve Body/Inline Body
102: Compression Spring
103: Fluid Inlet
104: Inlet Insert
105: Outlet Insert
106: Traveling Piston
107: O-Ring Seals
108: Airflow Pathway
109: Union Adapter
110: Alignment Tabs
111: Purge Pathway
112: RFID Chip and Antenna
113: ASD Pipe
114: Union Fitting
115: ASD Unit
116: Piston Default Position. Air Pathway Open
117: Purge Pathway Closed from Airflow Pathway
118: Closed Position. Air Pathway Closed
119: Purge Pathway Open for Purge Operation
120: Optional Inline Filter placed either within inlet or outlet

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

FIG. 1 is an external view of a self-actuating inline check valve assembly installed within an air sampling pipe network, illustrating the valve body positioned between an air sampling smoke detection (ASD) pipe and an ASD unit, and further showing a fluid inlet for receiving pressurized fluid.

FIG. 2 is a sectional view of the valve assembly showing internal components including a valve body, compression spring, traveling piston, O-ring seals, inlet insert, outlet insert, airflow pathway, purge pathway, alignment tabs, union adapter, and an RFID chip and antenna.

FIG. 3 is a sectional view of the valve assembly illustrating the traveling piston in a default position in which the airflow pathway is open and the purge pathway is closed from the airflow pathway.

FIG. 4 is a sectional view of the valve assembly illustrating the traveling piston in a purge position in which the airflow pathway is closed and the purge pathway is open for purge operation.

### DETAILED DESCRIPTION OF THE INVENTION

In the following detailed description of exemplary embodiments of the invention, reference is made to the accompanying drawings (where like numbers represent like elements), which form a part hereof, and in which is shown by way of illustration specific exemplary embodiments in which the invention may be practiced. These embodiments are described in sufficient detail to enable those skilled in the art to practice the invention, but other embodiments may be utilized and logical, mechanical, electrical, and other changes may be made without departing from the scope of the present invention. The following detailed description is, therefore, not to be taken in a limiting sense, and the scope of the present invention is defined only by the appended claims.

In the following description, numerous specific details are set forth to provide a thorough understanding of the invention. However, it is understood that the invention may be practiced without these specific details. In other instances, well-known structures and techniques known to one of ordinary skill in the art have not been shown in detail in order not to obscure the invention. Referring to the figures, it is possible to see the various major elements constituting the apparatus of the present invention.

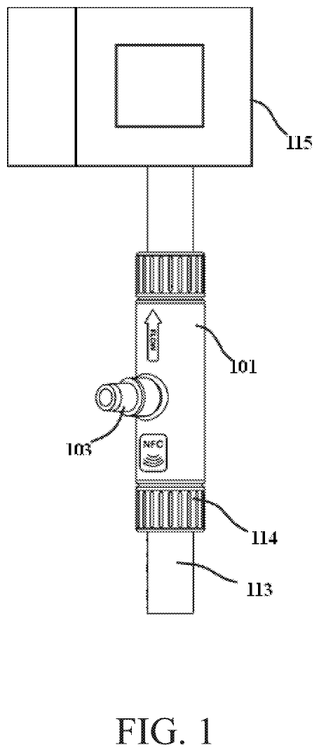

Referring initially to FIG. 1, an external view of a self-actuating inline check valve assembly is illustrated as installed within an air sampling pipe network. The valve assembly includes a valve body (101) configured for inline installation between an air sampling smoke detection (ASD) pipe (113) and an ASD unit (115). The valve body (101) is connected using union fittings (114), enabling installation and removal within existing piping systems. A fluid inlet (103) is provided on the valve body (101) to allow introduction of pressurized fluid for purge operations.

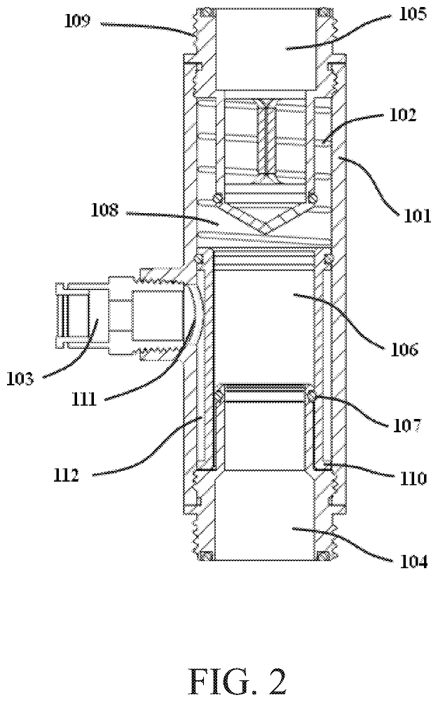

Referring now to FIG. 2, a sectional view of the valve assembly is shown illustrating internal components of the valve body (101). The valve body (101) defines an internal chamber in which a traveling piston (106) is disposed. The traveling piston (106) is biased by a spring (102), which in certain embodiments comprises a compression spring, toward a default position. The piston (106) includes one or more O-ring seals (107) configured to maintain system integrity and prevent leakage within the valve assembly. The valve body (101) further includes an inlet insert (104) and an outlet insert (105), which define respective fluid interfaces for airflow through the assembly.

An airflow pathway (108) is defined through the valve body (101) for normal operation, allowing air from the ASD pipe (113) to pass through the valve body (101) to the ASD unit (115). A purge pathway (111) is also defined within the valve body (101), which is selectively opened during purge operations. The traveling piston (106) is guided within the valve body (101) by alignment tabs (110) to maintain proper positioning within the internal chamber. A union adapter (109) may be provided to facilitate connection within the piping system. Additionally, the valve assembly may include an RFID chip and antenna (112) for storing and tracking system data. In certain embodiments, both the airflow pathway and the purge pathway are defined within the valve body and are simultaneously controlled by movement of the traveling piston.

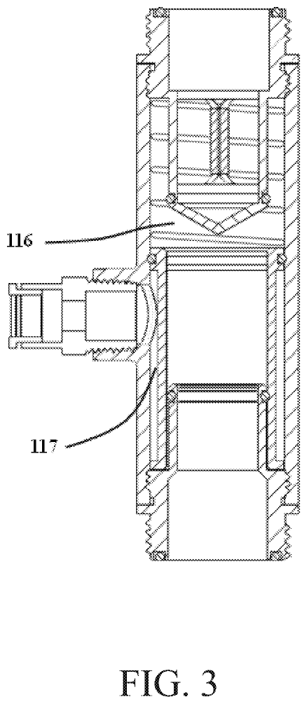

Referring to FIG. 3, the valve assembly is illustrated in a default operating condition. In this configuration, the compression spring (102) biases the traveling piston (106) into a default position (116) in which the airflow pathway (108) remains open, allowing air to flow freely from the ASD pipe (113) to the ASD unit (115). In this default condition, the purge pathway (111) is closed from the airflow pathway, as indicated by reference (117), thereby preventing purge fluid or air from entering the airflow pathway during normal operation.

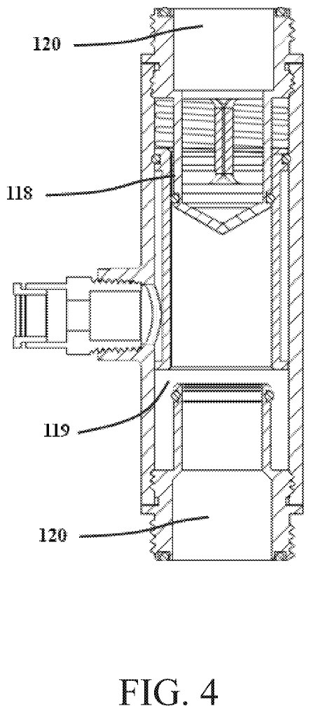

Referring to FIG. 4, the valve assembly is illustrated in a purge operating condition. When pressurized fluid is introduced through the fluid inlet (103) at or above a threshold pressure sufficient to overcome the bias of the compression spring (102), the traveling piston (106) is displaced within the valve body (101). In this translated position, the airflow pathway (108) is closed, as indicated by reference (118), thereby isolating the ASD unit (115) from the pipe network, and the purge pathway (111) is opened, as indicated by reference (119), such that movement of the traveling piston simultaneously closes the airflow pathway and opens the purge pathway. This allows pressurized fluid to pass through the valve body (101) and into the ASD pipe (113) to perform purging of the pipe network and sample holes. In certain embodiments, the traveling piston is configured such that movement between the default position and the purge position fully closes the airflow pathway and fully opens the purge pathway.

Upon removal of the applied pressure at the fluid inlet (103), the compression spring (102) returns the traveling piston (106) to the default position (116), thereby reopening the airflow pathway (108) and closing the purge pathway (111). This automatic return provides a fail-safe operation in which normal airflow to the ASD unit (115) is restored without manual or electronic intervention. As used herein, ‘fail-safe’ refers to a configuration in which the valve assembly automatically returns to a default airflow condition upon removal of actuation pressure.

The valve assembly may further include an optional inline filter (120), which may be positioned within either the inlet insert (104) or the outlet insert (105). The optional inline filter (120) provides filtering functionality during operation of the valve assembly.

The valve assembly is configured to operate within air sampling smoke detection systems, including environments such as data centers, warehouses, cold storage, industrial facilities, and commercial facilities. During operation, the valve assembly enables automatic purging of pipe networks and sample holes without requiring manual valve manipulation or electrically actuated components.

The valve body (101) and associated components may be constructed from materials compatible with air sampling systems, including ABS plastic, CPVC, stainless steel, or other corrosion- and pressure-resistant polymers or alloys. The design is scalable and adaptable to different pipe diameters and environmental conditions.

In operation, the valve assembly provides a self-actuating, fluid-actuated mechanism that isolates the ASD unit (115) during purge operations and restores airflow upon completion of purging. The configuration prevents improper system configuration associated with manual valves and eliminates the need for electrically actuated valves, thereby reducing system complexity and cost while maintaining system integrity and enabling automated maintenance.

In some embodiments, the inclusion of the RFID chip and antenna (112) enables tracking of system data, maintenance activity, or inventory information associated with the valve assembly.

Alternative embodiments may include the addition of pressure sensors to confirm purge operations, electronic actuators for dual-mode operation, integrated diagnostics or tamper detection, or alternative actuation mechanisms, including diaphragm-based actuators, membrane systems, or other moveable flow control elements configured to selectively control fluid pathways in a manner consistent with switching functionality described herein.

Thus, it is appreciated that the optimum dimensional relationships for the parts of the invention, to include variation in size, materials, shape, form, function, and manner of operation, assembly, and use, are deemed readily apparent and obvious to one of ordinary skill in the art, and all equivalent relationships to those illustrated in the drawings and described in the above description are intended to be encompassed by the present invention.

Furthermore, other areas of art may benefit from this method and adjustments to the design are anticipated. Thus, the scope of the invention should be determined by the appended claims and their legal equivalents, rather than by the examples given.

1. A self-actuating check valve assembly comprising:
a valve body configured for inline installation within an air sampling pipe network between an air sampling pipe and an air sampling smoke detection (ASD) unit;
a traveling piston disposed within the valve body;
a spring configured to bias the traveling piston to a default position;
a fluid inlet configured to receive pressurized fluid;
an airflow pathway defined through the valve body and configured to permit airflow to the ASD unit when the traveling piston is in the default position; and
a purge pathway defined through the valve body;
wherein the airflow pathway and the purge pathway are both defined within the valve body and are simultaneously controlled by movement of the traveling piston;
wherein, upon introduction of pressurized fluid through the fluid inlet at or above a threshold pressure, the traveling piston is displaced against the spring from the default position to a purge position, wherein movement of the traveling piston from the default position to the purge position simultaneously:
closes the airflow pathway to the ASD unit, and
opens the purge pathway to direct the pressurized fluid through the air sampling pipe network, thereby isolating the ASD unit; and
wherein, upon removal of the pressurized fluid, the spring automatically returns the traveling piston to the default position to reopen the airflow pathway to the ASD unit.

2. The assembly of claim 1, wherein the valve body is configured for connection to the air sampling pipe and the ASD unit using union fittings.

3. The assembly of claim 1, further comprising an inlet insert and an outlet insert disposed within the valve body.

4. The assembly of claim 1, wherein the traveling piston includes one or more O-ring seals configured to maintain system integrity.

5. The assembly of claim 1, further comprising alignment tabs configured to guide movement of the traveling piston within the valve body.

6. The assembly of claim 1, further comprising a union adapter configured to facilitate connection of the valve body within the air sampling pipe network.

7. The assembly of claim 1, further comprising an RFID chip and antenna configured to store and track system data.

8. The assembly of claim 1, further comprising an inline filter disposed within at least one of an inlet or an outlet of the valve body.

9. The assembly of claim 1, wherein the airflow pathway is open and the purge pathway is closed when the traveling piston is in the default position.

10. The assembly of claim 1, wherein the airflow pathway is closed and the purge pathway is open when the traveling piston is in the purge position.

11. A method of purging an air sampling pipe network comprising:
providing a valve assembly installed inline between an air sampling pipe and an air sampling smoke detection (ASD) unit, the valve assembly including a valve body, a traveling piston, a spring, an airflow pathway, and a purge pathway;
permitting airflow through the airflow pathway to the ASD unit when the traveling piston is in a default position;
introducing pressurized fluid into the valve assembly through a fluid inlet;
displacing the traveling piston against the spring to a purge position;
closing the airflow pathway to isolate the ASD unit;
opening the purge pathway;
directing the pressurized fluid through the purge pathway into the air sampling pipe network to perform a purge operation; and
removing the pressurized fluid to allow the spring to return the traveling piston to the default position, thereby reopening the airflow pathway.

12. The method of claim 11, wherein the purge operation removes accumulated contaminants from the air sampling pipe network.

13. The method of claim 11, wherein the purge operation removes moisture or ice from the air sampling pipe network.

14. The method of claim 11, further comprising maintaining a seal within the valve assembly using one or more O-ring seals during operation.

15. The method of claim 11, further comprising guiding movement of the traveling piston using alignment tabs within the valve body.

16. A self-actuating inline valve assembly, comprising:
a valve body configured to be installed inline within a fluid conduit system between an upstream conduit and a downstream device;
a piston disposed within the valve body and movable between a first position and a second position;
a spring configured to bias the piston toward the first position;
a port configured to receive pressurized fluid;
a first flow path configured to allow fluid flow to the downstream device when the piston is in the first position; and
a second flow path configured to allow purge flow through the valve body when the piston is in the second position;
wherein introduction of pressurized fluid into the port displaces the piston from the first position to the second position such that movement of the piston simultaneously closes the first flow path and opens the second flow path to direct the pressurized fluid through the conduit system; and
wherein removal of the pressurized fluid allows the spring to return the piston to the first position such that movement of the piston reopens the first flow path and closes the second flow path.

17. The assembly of claim 16, wherein the valve body includes connection interfaces for inline installation within an air sampling pipe network.

18. The assembly of claim 16, wherein the piston includes sealing elements configured to prevent leakage between the first flow path and the second flow path.

19. The assembly of claim 16, further comprising a tracking component configured to store system data.

20. The assembly of claim 16, further comprising a filter disposed within the valve body.
